(19) United States
(12) Patent Application Publication
(10) Pub. No.: US 2023/0070807 A1
(43) Pub. Date: Mar. 9, 2023

(54) SYSTEMS AND METHODS FOR ASSESSING COLONOSCOPY PREPARATION

(71) Applicant: DARK CANYON LABORATORIES, LLC, Rapid City, SD (US)

(72) Inventors: Dale R. Bachwich, Rapid City, SD (US); Vera O. Kowal, Rapid City, SD (US)

(21) Appl. No.: 17/792,933

(22) PCT Filed: Jan. 14, 2021
(86) PCT No.: PCT/US21/13433

§ 371 Date: Jul. 14, 2022

Related U.S. Application Data
(60) Provisional application No. 62/961,482, filed on Jan. 15, 2020.

(51) Int. Cl. A61B 10/00; G01N 21/88; G06T 7/00; G06T 7/90; G06T 7/40

(52) U.S. Cl. CPC A61B 10/0038; G01N 21/8851; G06T 7/0002; G06T 7/90; G06T 7/40; G01N 2021/8887

(57) ABSTRACT

The present disclosure relates to systems and methods for assessing the status of colon cleansing. In particular, the present disclosure relates to systems and methods to determine completion of colon preparation in subjects prior to a gastrointestinal procedure in real time.

### CROSS-REFERENCE TO RELATED APPLICATIONS

This application claims the benefit of U.S. Provisional Application No. 62/961,482, filed Jan. 15, 2020, the entire contents of which is incorporated herein by reference.

### FIELD

The present disclosure relates to systems and methods for assessing the status of colon cleansing. In particular, the present disclosure relates to systems and methods to determine completion of colon preparation in subjects, e.g., in real-time, prior to a gastrointestinal procedure.

### BACKGROUND

Millions of colonoscopy procedures are performed in a year, most often in an outpatient setting. Preparation prior to the day of the procedure is required to clean out the bowel contents to allow examination of the intestine walls. The preparation is typically completed at home by the patient usually over 2 days. In general, colonoscopy prep medications are difficult for patients to tolerate and patients are acutely interested in minimizing the amount of prep they drink prior to their colonoscopy.

Current colonoscopy prep medications are dosed “one size fits all.” However, patients with certain risk factors, including but not limited to a history of poor colon prep on previous colonoscopies, male gender, obesity, hypertension, diabetes, constipation, drugs that affect motility (narcotics, tri-cyclic antidepressants), and low educational level, are at higher risk of having an inadequate or suboptimal prep. Overall, approximately 40-70% of all colonoscopy patients have at least one recognizable risk factor for a suboptimal prep. Unfortunately, no one has been able to construct a schema to allow individualized dosing of the prep with any useful accuracy.

Assessment of clarity and color of effluent and amount of particulate matter is key to determining the success or progress of the colon prep. However, patients are left to their own devices to assess if their prep is working properly or not. Presently, there are no readily accessible standards that allow patients to determine their prep effectiveness or completion. Further, even if these standards were accessible by patients, it remains to be seen as to whether these standards are effective when patients attempt to use them at home. It is not unusual for patients to place emergency phone calls to their physicians during the night to ask about certain symptoms they may be experiencing and to ask if they should continue the prep. It is also not unusual for patients experiencing such symptoms to prematurely terminate their prep under the mistaken conclusion that the prep, in their untrained estimation, is adequate.

Two research studies have documented that patients' own descriptions of their own fecal effluent are very inaccurate and cannot be relied upon in most instances to ascertain whether they need additional preparation (Harewood G C, et al., Am J Gastroenterol 2004; 99:839-43 and Fatima H, et al., Gastrointest Endosc 2010:71:1244-1252 e2, incorporated herein by reference in their entirety). The only situation where the patient is accurate, albeit only 50% of time, is when they describe their own effluent as brown and having solid material present, signifying an inadequate prep. However, without exception, patients prefer to prep in the privacy of their home and are reluctant to undergo additional prep at an endoscopy center or hospital using a public restroom.

Inadequate or incomplete preparation can result in a cancelled case or the necessity for an earlier repeat examination. Approximately 15-25% of all colonoscopy cases are inadequate. A stated goal of GI Societies is to reduce inadequate prep rates to less than 10-15%. An additional fraction, perhaps as high as 50% of preps, are less than excellent. Sub-optimal, or less than excellent, preparation requires an average of 4-5 minutes per case cleaning the colon to render the prep adequate prior to inspection. This can be as much as 10-15 minutes in some cases. This unnecessarily exposes the patient to the risk of prolonged anesthesia. Even with this cleansing, not all suboptimal preps can be salvaged. Current GI Society guidelines instruct gastroenterologists to repeat colonoscopy within one year if the prep quality is borderline, rather than a repeat colonoscopy in ten years if the bowel preparation is of good quality. For patients with a completely failed prep, which precludes completion of the exam, it is recommended that they repeat their prep and return as soon as possible for a reattempt at colonoscopy. Studies have documented that a third of such patients with a failed prep do not return for another. Inadequate and suboptimal colon preparations make colonoscopies less convenient, less cost-effective, and less accurate, resulting in more missed cancers and polyps.

### SUMMARY

Disclosed herein are methods for assessing the status of a colon preparation in a subject comprising capturing an image of a bowel effluent in a toilet with an imaging device and assessing the image for: one or more aspects of the bowel effluent including color of effluent, clarity of effluent, presence of particulate matter or solid material, density of particulate matter or solid material, texture of the effluent, or combinations thereof; observable edges or contrasts in the toilet; or any combination thereof. In some embodiments, the methods may further comprise one or each of capturing a background image of the toilet, and loading the image, and optionally the background image, into an information processing component from the imaging device. In some embodiments, the methods further comprise adding a flushable marker to the toilet. In some embodiments, the methods further comprise adding an antifoaming agent to the toilet.

Also disclosed herein are systems for assessing the status of a colon preparation in a subject comprising an imaging device, a communication component, an information processing component configured to analyze data from two or more images of bowel effluent, and a protocol component. In some embodiments, the imaging device is integrated into the information processing component. In some embodiments, the information processing component is a tablet computer, a portable computer, or a wearable computer. In some embodiments, the information processing component is in communication with a clinician device in contact with a clinician.

Other aspects and embodiments of the disclosure will be apparent in light of the following detailed description and accompanying figures.

### BRIEF DESCRIPTION OF THE DRAWINGS

FIG. 1 is a flow diagram illustrating select embodiments of the methods for assessing the status of a colon preparation in a subject.

FIG. 2 is a flow diagram illustrating exemplary feedback to the artificial intelligence component for machine learning.

FIGS. 3A-3E are exemplary toilet bowl images during colon preparation. FIG. 3A is an exemplary baseline toilet bowl image. FIGS. 3B-3D are exemplary images throughout colon preparation taken at an early stage (FIG. 3B), midway through preparation (FIG. 3C), and nearing the end of the preparation (FIG. 3D). FIG. 3E is an exemplary image at the end of the preparation.

FIGS. 4A-4I are exemplary toilet bowl images during colon preparation. FIGS. 4A, 4D, and 4G, are a raw image, an image resulting from using edge detection, and an image resulting from using red pixel intensity, respectively, of an exemplary baseline image, as the preparation commenced. FIGS. 4B, 4E, and 4H, are a raw image, an image resulting from using edge detection, and an image resulting from using red pixel intensity, respectively, of an exemplary final image where preparation was still deemed incomplete (Not Ready). FIGS. 4C, 4F, and 4I, are a raw image, an image resulting from using edge detection, and an image resulting from using red pixel intensity, respectively, of an earliest image where the preparation was sufficient (Ready), based on the judgements of a colonoscopy preparation expert.

FIG. 5 is an image of exemplary marker capsules.

FIGS. 6A-6D are images showing the effect of a marker capsule on edge detection. FIGS. 6A and 6B are images of a dark toilet drain at baseline without and with the use of a marker capsule.

FIGS. 6C and 6D are images after applying edge detection to the images of FIGS. 6A and 6B, respectively.

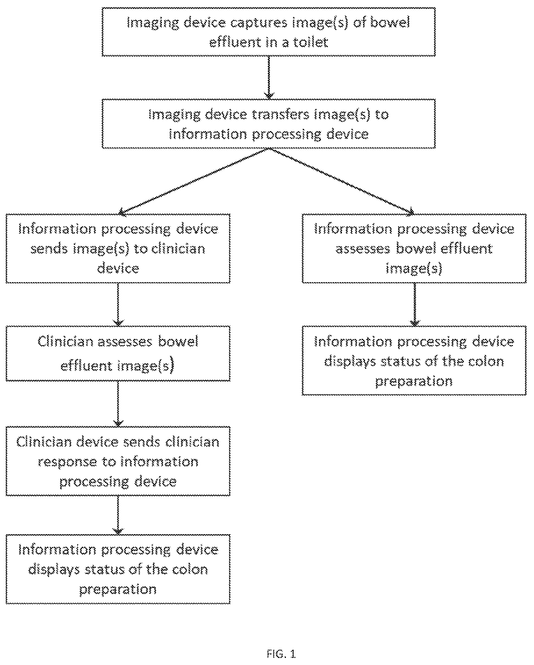

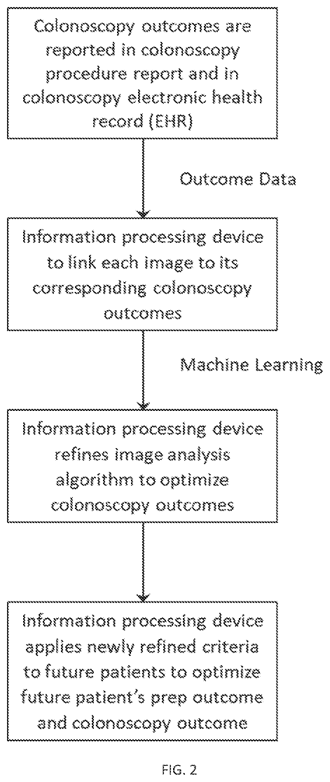

### DETAILED DESCRIPTION

Section headings as used in this section and the entire disclosure herein are merely for organizational purposes and are not intended to be limiting.

### 1. DEFINITIONS

The terms “comprise(s).” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “processor” and “central processing unit” or “CPU” are used interchangeably and refer to a device that is able to read a program from a computer memory (e.g., ROM or other computer memory) and perform a set of steps according to the program.

As used herein, the terms “computer memory” and “computer memory device” refer to any storage media readable by a computer processor. Examples of computer memory include, but are not limited to, RAM, ROM, computer chips, digital video discs (DVD), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks. The computer readable medium may be non-transitory or include a device of system that is not a transitory signal.

As used herein, the term “in electronic communication” refers to electrical devices (e.g., computers, processors, etc.) that are configured to communicate with one another through direct or indirect signaling.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human). In one embodiment of the methods and systems provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. ASSESSING COLON PREPARATION

The present disclosure provides methods and systems to assess the status of a colon preparation in a subject. In some embodiments, the methods and system assess the status (e.g., determine end-point) of a colon preparation in a subject in real-time.

Colon preparation is employed by a patient to cleanse their colon and digestive tract prior to a colon-related or gastrointestinal medical procedure, including, for example, colonoscopy, capsule endoscopy, single- and double-balloon enteroscopy, endoluminal gastroplication, endoscopic ultrasound (EUS), esophagogastroduodenoscopy (EGD), endoscopic retrograde cholangiopancreatography (ERCP), esophageal pH exam, flexible sigmoidoscopy (Flex Sig), hydrogen breath test, liver biopsy, percutaneous endoscopic gastrostomy (PEG), biofeedback for anorectal dysfunction, intraoperative radiation therapy (TORT), diagnostic GI radiology, including barium enema, CT colonography, CT enterography, MR colonography, and MR enterography; surgical procedures including, but not limited to laparoscopic cholecystectomy (Gallbladder Removal), colectomy, hysterectomy, hemorrhoidectomy, hemiorrhaphy, and NOTES (natural orifice transluminal endoscopic surgery).

There are a number of ways to cleanse the colon, each with advantages and disadvantages. Usually, patients are provided medications in the form of solutions and/or pills to take for up to two days prior to surgery. Purgatives can be based on magnesium salts, sodium phosphate, or buffered saline solution with polyethylene glycol (PEG). Bisacodyl is sometimes added to the regimen to stimulate colonic motility. The systems and methods disclosed herein may be incorporated with any type of colon preparation or gastrointestinal medical procedure. Suitable nonlimiting examples of colon preparations useful in the methods and systems disclosed herein include those disclosed in U.S. Pat. Nos. 8,778,306 and 10,555,970 and U.S. patent application Ser. No. 16/786,541, each of which is herein incorporated by reference in its entirety.

There are a number of aspects of bowel effluent used to assess cleansing of the colon. In some embodiments, assessment aspects include, but are not limited to, color of effluent, clarity of effluent, texture of the effluent presence of particulate matter or solid material in the effluent, and density of particulate matter or solid material in the effluent.

In some embodiments, considerations in assessing the image may include if any edges at the bottom of a toilet bowl are visible, if any edges from toilet paper beneath surface are visible, if there is a color gradient at toilet bowl edges descending to the bottom of the bowl, or if there is a contrast gradient with a flushable marker. In some embodiments, the observable edges or contrasts in the toilet comprise edges or contrasts along bottom of toilet bowl, edges or contrasts from the flushable marker, or a combination thereof. Specific examples of select aspects may be found in: So, H., et al., Intest Res 2015: 13(2): 153-159 and Fatima, H., et al., Clinical Endoscopy 2010; 71(7): 1244-1252, incorporated herein by reference in their entirety.

In some embodiments, the systems and methods comprise one or more or each of: an imaging component, a communication component, an information processing component, and a protocol component. In some embodiments, the methods further comprise adding a flushable marker and/or an anti-foaming agent to the toilet. In some embodiments, the methods further comprise a clinician device. Illustrative embodiments of each of these components, their integration into the system, and their use in the methods is described below.

a) Imaging Device

In some embodiments, the systems and methods comprise an imaging device. The imaging device facilitates capture of an image of bowel effluent in a toilet, and optionally an image of the toilet without bowel effluent, preferably taken immediately beforehand.

The imagining device may include, for example, a camera, a cellular phone with an embedded camera, or a computer. The imaging device can comprise hardware such as the image sensor (e.g., CCD (charge coupled device), CMOS (complementary metal oxide semiconductor), etc.) and software for operating the image sensor and/or the imaging component. In some embodiments, the imaging device is incorporated or embedded in one of the other components of the system (e.g., the information processing component). In some embodiments, an imaging device is integrated into a toilet and automatically, or upon activation by a user, captures one or more images before and after (or during) generation of a bowel effluent. In some embodiments, sensors detect a toilet-related event (e.g., lifting of a lid, contact or depression of a handle, reduction in light due to movement of a person over the toilet, passage of effluent material into the toilet, etc.) in order to trigger the timing of image capture.

b) Communication Component

In some embodiments, the systems and methods comprise a communication component. In some embodiments, the communication component communicates information from the imaging device to an information processing component. In some embodiments, the communication component communicates information to or from the information processing component (e.g., to or from the imaging device or a clinician device). In some embodiments, the communication component communicates information from any one component of the system (e.g., an imaging component), to any other component of the system, between two sub-components of the system, or between a component outside of the system (e.g., a clinician device or an external device, for example, a wearable physiological tracker)).

In some embodiments, a portion or all of the communication component is wired. For example, in some embodiments, the communication component comprises wires or cables connecting the imaging device directly or indirectly to an information processing component (e.g., computer, a computer processor within a phone, etc.).

In some embodiments, a portion of or the entire communication component is wireless. Any desired wireless communication technology may be employed, including but not limited to, electromagnetic wireless telecommunications (e.g., wireless networking, cellular, satellite), and electromagnetic induction (such as light, magnetic, or electric fields or the use of sound). Where wireless networks are employed, any desired protocol can be used (e.g., ZigBee, EnOcean, Personal area networks, Bluetooth, TransferJet, ultra-wideband).

In some embodiments, the imaging device comprises a wireless communication component such that signal generated from the imaging device is transmitted to an information processing component wirelessly in a HIPAA-compliant fashion. In some embodiments, the communication component communicates information comprising images and/or instructions to the information processing component.

c) Information Processing Component

In some embodiments, the systems and methods comprise an information processing component. The information processing component can provide a variety of functions, including but not limited to: receiving and processing images generated from the imaging device; receiving and processing data generated from an external device; displaying information to a subject; displaying instructions to a subject; storing information; storing, transmitting, executing, and/or displaying protocols: and presenting alarms.

In some embodiments, the information processing component comprises one or more of a computer processor, computer readable medium, and software. Any of a variety of computing devices may be used as the information processing component, including but not limited to, a desktop computer, a mainframe computer, a laptop computer, a personal digital assistant (PDA), a portable computer (e.g., mobile devices such as telephones), a tablet computer (e.g., standard tablets, slates, mini tablets, phablets, customer handheld devices), and a wearable computer (e.g., eyeglass, wristwatch, clothing, helmet, etc.). In select embodiments, the information processing component comprises a tablet computer, a portable computer, or a wearable computer.

In some embodiments, the information processing component or a device in electronic communication with the information processing component (e.g., a video monitor) comprises a display. In some embodiments, the display displays textual and/or graphical information to a user (e.g., a subject completing a colon preparation). In some embodiments, the display is a touchscreen display, permitting the user to select and manage system functions via a graphical interface. In some embodiments, audio information is conveyed (e.g., via speakers, headphones, etc.).

In some embodiments, the display displays information to the user related to instructions for or status of the colon preparation. In some embodiments, the interface displays status identifiers (e.g., ready, good progress, pause, not ready). In some embodiments, the interface provides an additional graphical interface of status (e.g., stop light indications).

In some embodiments, the interface further provides textual instructions (e.g., stop taking prep, drink only clear liquids until procedure, wait 30 minutes, take another image of effluent, take another dose of prep medications). In some embodiments, the interface provides an additional graphical interface of instructions completed (e.g., thumbs up/thumbs down or check marks). The user interface may also provide step by step instructions of setting up, using, and managing the system.

In some embodiments, the information processing component, or a device in electronic communication with the information processing component, comprises a networking component. The networking component receives and/or transmits information to the communication component.

In some embodiments, the information processing component comprises a database containing protocols, subject data, historic data, or other desired information. The protocols, subject data, historic data, or other desired information may be provided by computer processor, computer readable medium, software, or be available through a web-based platform via a web browser across the internet.

In some embodiments, the information processing component is in communication with a clinician device in contact with a clinician. In some embodiments, the communication with the clinician device is via the networking component and/or the communication component.

In some embodiments, the information processing component comprises an artificial intelligence component (e.g., embodied in software running on the processor). In some embodiments, the artificial intelligence component alters stored protocols in response to instructions received from another component of the system (e.g., protocol component) or from another device (e.g., clinician device).

In some embodiments, the artificial intelligence component comprises image analysis software. In some embodiments the image analysis software evaluates the image obtained by the imaging component. The image analysis software may assess the status of the bowel effluent. In some embodiments, the image analysis software may assess the observable edges or contrasts (e.g., a flushable maker) on the bottom of or in the toilet bowl.

The image analysis software may include the ability to transform the images provided by the subject, creating enhanced images which may include highlighting, coloring, emphasis or de-emphasis of detail, digital filtering, among many other potential transformations. For example, the image analysis software may subtract out the background image without the bowel effluent or subtract out other foreign objects or abnormalities, such as toilet paper, glare, or shadows, from the bowel effluent image. The image analysis software may transform the image to align and correct the image to resemble the background image in brightness/contrast, size, and orientation (e.g., pan, tilt and rotation). The imaging software could be custom designed, licensed from third parties, or even commercially available software. Multiple software programs may be utilized together in order to fully analyze the image.

The image analysis software may also generate results and/or reports based on the analysis of the image. In some embodiments, the artificial intelligence component alters protocols or displays instructions based on the image analysis evaluation of the bowel effluent (e.g., utilizing a machine learning process). In some embodiments, the artificial intelligence component provides real-time feedback of the status of the colon cleanse to the subject based on the image analysis software. Consequently, the artificial intelligence component facilitates customization of protocols and prep medication dosing (e.g., increase or decrease in amount or frequency, continuation of dosing regimen, termination of dosing regimen) in real-time, based on active monitoring of the images unlike convention preparations utilizing a universal approach without a form of monitoring. In some embodiments, the artificial intelligence component instructs the information processing component to report the image results to a clinician device.

The baseline toilet bowl image is analyzed with regard to lighting quality and focus. Additional processing may occur, including, for example: identification of the meniscus: determination of uniformity of lighting across the field within the meniscus; determination of the level of focus of edges within the area defined by the meniscus (the region of interest); determination of the color of the toilet bowl outside of the meniscus in comparison to the color just inside the meniscus; subtraction of the bowl color from the image in those instances when the toilet bowl color is not white; and identification of the greatest color variation between segments of the image just inside the meniscus and other sites within the region of interest, excluding edges.

Subsequent images are analyzed comparing the baseline image with the newest image for focus, color analysis, edge analysis, visual texture and time analysis. Each image is analyzed as the baseline image to identify the meniscus and determine if the overall image is in focus. In some embodiments, the methods and systems comprise addition of an anti-foaming agent, such as simethicone and the like, to the toilet to allow for better focus, color analysis, edge analysis, and visual texture during image analysis.

Color analysis may include: subtraction of the baseline image color from the image being analyzed, detection and determination of a color change in the region of interest; determination of the colors of the textons (particular objects creating texture) in the region of interest: and evaluation of the new image data using algorithms defining acceptable and optimal values for color of the toilet water and of the textons. In an example, but not limited by this example, the red color pixel intensity of the water in the grayscale image should have values of 125 to 255 to be acceptable. Red color pixel values of water below 125 indicate inadequate preparation in this example.

Edge analysis includes determination if any of the previously defined edges within the area defined by the meniscus (the region of interest) still in view and if there any new edges within the region of interest. In some embodiments, edge analysis includes assessing observable contrast or edges from a flushable marker. Flushable markers include any agent or object useful in providing contrast to the image of the toilet, including, but not limited to, capsules, tablets, caplets, or pellets of any variety of sizes and shapes made of a flushable material in a color contrasting the toilet and/or effluent.

Visual texture analysis includes determination of the sizes of the textons within the region of interest and the homogeneity in the sizes of the textons within the region of interest and evaluation of any observable three-dimensional texture within the region of interest.

Once a second subsequent image is available, each image will be analyzed to determine if there was a progressive change in edge improvement, color dilution, or texton size/homogeneity from the previous image(s).

Following the colon preparation and procedure, the artificial intelligence component will go through a machine learning process, both supervised and unsupervised. The machine learning process provides feedback the artificial intelligence uses to alter the protocol component. The endoscopy electronic health record (EHR) provides images and other data to the software; the software uses the images coupled to other outcome parameters to provide feedback to the system. The feedback includes, for example, success of preparation (Boston Bowel Preparation Scale (BBPS) 6 or better, with a 2 or better in every segment); level of intra-procedural work required to make preparation successful; quality of the preparation (high quality is considered BBPS 8 or better); quantity of intra-procedural work necessary to make preparation high quality; completion of colonoscopy; detection and quantification of adenomatous polyps; detection and quantification of serrated polyps; detection of colon cancer.

As such, the machine learning process aids in the creation of thresholds for color, turbidity (ability to detect edge), and visual texture for both size and color of textons for each of the following conditions: successful preparation, high quality preparation, successful prep with limited work, where the user may specify the work limit: high quality preparation with limited work, where the user may specify the work limit; unsuccessful prep: and preparation complicated by bleeding event.

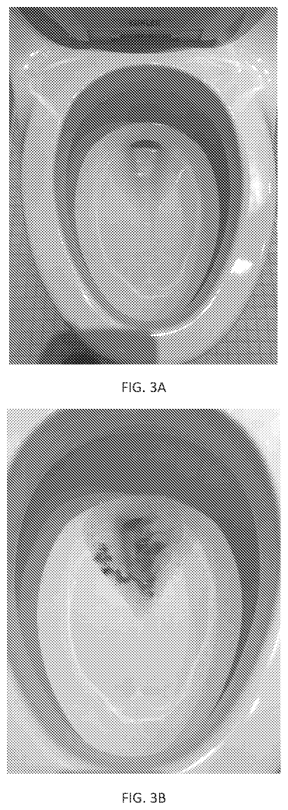

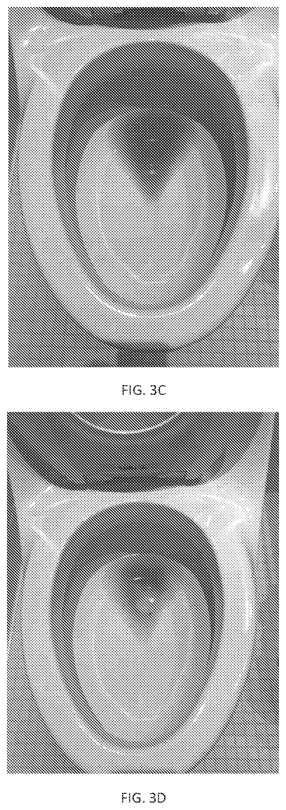

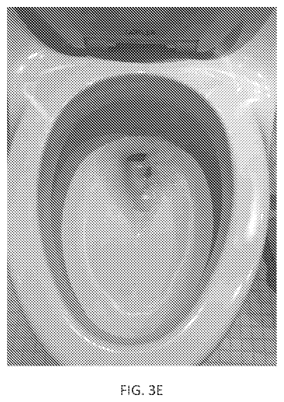

Shown in FIGS. 3A-E is an example of toilet bowl images from a preparation, using artificial stool. FIG. 3A shows a high-quality baseline toilet bowl image. An image early in the preparation, FIG. 3B, shows textons (particular matter) too large to be acceptable. Midway through the preparation, the textons are much smaller however, no edges seen in the baseline photo can be observed and the color criteria have not been met (FIG. 3C). Nearing the end of the preparation (FIG. 3D), even though the fluid is clearer than FIG. 3C, no edges seen in the baseline image can be discerned, the textons other than toilet paper are larger than 2 mm, and the color has a Green intensity of 108, below a criteria threshold of 160. Upon completion of the preparation (FIG. 3E), edges from the baseline image can be easily seen, no textons other than toilet paper are visible, and the color is acceptable with a Green intensity of 180.

The disclosure also provides a pre-procedure preparation scale and methods of creating and using a pre-procedure preparation scale. In some embodiments, the machine learning process aids in the creation of a pre-procedure preparation scale and/or applies image analysis to classify or score the image based on a pre-procedure preparation scale. In some embodiments, the pre-procedure preparation scale is based on analysis of bowel effluent (e.g., color of effluent, clarity of effluent, presence of particulate matter or solid material, density of particulate matter or solid material, texture of the effluent, or combinations thereof) and/or observable edges or contrasts (e.g., from a flushable marker). The scale may be used to correlate the preparation or the status of the preparation with existing bowel cleanliness scales (e.g., Boston Bowel Preparation Scale, Aronchick Scale, Ottawa Bowel Preparation Scale, and Harefield Cleansing Scale). The threshold may be adjusted for the individual patient, the planned colonic procedure, or other clinical factors. For example, a standard colonoscopy may have a lower acceptable score than a wireless capsule colonoscopy which requires a very high level of cleanliness.

The pre-procedure preparation scale may be updated based on ongoing data collection, including, but not limited to colonoscopic video during examinations, programs that monitor and score colonic cleanliness intra-procedurally, existing bowel cleanliness scales including the Boston Bowel Preparation Scale. Aronchick Scale, Ottawa Bowel Preparation Scale, Harefield Cleansing Scale, and clinically relevant outcome measures. Such clinical measures include adenoma detection rate, cleansing time, and overall procedure time. Such scale updates may be performed manually, by supervised artificial intelligence, or by unsupervised artificial intelligence. The described validation and update process would be performed in compliance with applicable FDA regulations.

An exemplary pre-procedure preparation scale may use a numbered-scale as shown in Table 1. The threshold for an acceptable prep based on this scale may be in the range of 5 to 6, which likely correlates to a score of 6 on the Boston Bowel Preparation Scale. The scores of any exemplary scales can be validated in randomized, controlled, clinical trials before general use.

TABLE 1
Exemplary Pre-Procedure Scale

| Score | Description |
|---|---|
| 0 | Solid stool with textons greater than 10 mm. |
| 1 | Solid stool with textons greater than 10 mm plus brown liquid. |
| 2 | Solid stool with textons less than 10 mm plus brown liquid. |
| 3 | Solid stool with textons less than 3 mm plus brown liquid. |
| 4 | Brown, opaque (no subsurface edges detected) liquid; no solid stool. |
| 5 | Brown, transparent liquid as discerned by X % of subsurface edge pixels detected; no |
| | solid stool. |
| 6 | Yellow-green, transparent liquid as discerned by (X + Y) % of subsurface edge pixels |
| | detected; no solid stool. |
| 7 | Yellow, transparent liquid as discerned by (X + Y) % of subsurface edge pixels detected; |
| | no solid stool. |
| 8 | Yellow, transparent liquid as discerned by (X + Y + Z) % of subsurface edge pixels |
| | detected; no solid stool. |
| 8B | Yellow, transparent liquid as discerned by (X + Y + Z) % of subsurface edge pixels |
| | detected; rare small bits of solid stool consistent wi th delayed colonic diverticulum |
| | release. |
| 9 | Yellow to colorless transparent liquid as discerned by (X + Y + Z) % of subsurface edge |
| | pixels detected; no solid stool. |
| 9B | Yellow, transparent liquid as discerned by (X + Y + Z) % of subsurface edge pixels |
| | detected; rare small bits of solid stool consistent with delayed colonic diverticulum |
| | release. |

X, Y, and Z are positive numbers, and X + Y + Z is less than or equal to 100. Color and intensity may be defined by the relative Red-Green-Blue pixel intensities.

In some embodiments, the information processing component, the imaging component, the communication component, or any combination thereof are contained within a single device (e.g., a tablet computer, a mobile phone, a wristwatch computer, etc.).

d) Protocol Component

In some embodiments, the systems and methods comprise a protocol component. The protocol component comprises instructions, typically embodied in software, for managing the methods and use of the system. The software comprises all non-transitory forms of software, or all those forms of software except those based on a transitory, propagating signal. In some embodiments, the protocol component is stored in a computer readable medium. In some embodiments, the protocol component is embodied in the information processing component.

In some embodiments, the protocol component directs the display of information. In some embodiments, the display comprises instructions (e.g., graphical, textual, audio, etc.) for use of the imaging component. For example, in some embodiments, the protocol component directs the subject to take an initial image or repeat an image if quality is not acceptable. In some embodiments, the protocol component displays information comprising tasks to be completed (e.g., how to take the medications, how to take the images, how to modify the prep based on instructions), knowledge of task completion, or status of colon preparation.

The protocol component may comprise a scheduling function, such that the protocol can provide instructions prior to starting the colon cleanse (e.g., low residue diet instructions 5 days prior to cleanse, consume only clear liquids evening before colon cleanse). In some embodiments, the protocol component may display instructions for the day of the medical procedure or provide background information or answers to frequently asked questions regarding the specific medical procedure.

In some embodiments, the format of the display is adjustable to accommodate any subject type, including those with impaired vision or hearing, impaired cognitive skills, color blindness, young age, varied language skills or knowledge, etc.

In some embodiments, the protocol component comprises data storage and management capabilities. Data storage includes data storage and management for individual subjects, for example, to monitor bowel effluent over time. In some embodiments, data storage also includes storage and management for multiple subjects, including, but not limited to, colonoscopy outcomes, bowel cleanliness at the time of exam, adenoma detection, intraprocedural cleansing effort required, relevant health history factors, and other relevant factors (e.g., body habitus, socio-demographic) that impact colon cleansing. These linked data may be used to make comparative analysis and/or improve artificial intelligence capabilities of the system which can then, in turn, be used to optimize the system and finally, applied to future patients using the system. This application of machine learning to optimize the management of future patients may be either supervised or unsupervised.

In some embodiments, the protocol component comprises specific colon cleansing protocols. In some embodiments, the protocol component allows selection of specific colon cleansing protocols (e.g., specific medications or specific medical procedures) such that the instructions are tailored to the specific colon cleansing protocol or image analyses are completed within the colon cleansing specifications for a specific medical procedure. The protocol may be selected by the subject following instructions by the clinician or pre-selected for the subject by the clinician through the integration with the clinician device. The protocols employed can be updated and optimized for any desired cleansing protocol or medical procedure by the clinician. In some embodiments, the protocol instructs the processor to report the image results to a clinician device. In some embodiments, the protocol may compare data from previous measurements to determine whether the subject has progressed with the colon cleansing. The specific colon cleansing protocol (e.g., amount and frequency of dosing of prep medications, maintenance of dosing beyond original dosing schedule, early termination of dosing schedule) may be customized during the preparation (e.g., by the clinician or based on image analysis) in real-time.

In some embodiments, the protocol component comprises a query function, such that the protocol component displays questions regarding symptoms or side effects that may be experienced during the colon preparation to the subject for a response. The query function may question the occurrence, absence, level or frequency of nausea, vomiting, thirst, light-headedness, abdominal pain, and the like. In some embodiments, the protocol component may adjust the protocol instructions (e.g., slow or pause colon cleansing protocol) based on the responses to the question(s). In some embodiments, the protocol component may send the responses to the information processing component which may adjust the instructions for the specific colon cleansing protocol based on the answers given to the questions regarding symptoms or side effects. Alternatively, the responses may be sent to the clinician device for review by the clinician and potential subsequent protocol adjustment. In some embodiments, the query function may instruct a patient to capture data regarding physiological parameters (e.g., temperature, heart rate, and the like) from a connected device as described elsewhere herein (e.g., wearable devices, clothing with embedded monitoring devices), an external device (e.g., home-based devices, such as thermometers, blood sugar monitoring devices, home audio monitoring equipment (e.g., Siri and Alexa), a cell phone app which enables smartphones to monitor physiologic or other parameters directly or indirectly by linking to afferent sensing hardware, toilet-based sensors, or the like. In some embodiments, the information processing component may detect an anomaly or side-effect from a connected external device and trigger the query function within the protocol component. For example, the information processing component may detect tachycardia from a wearable device and trigger the query function of the protocol component to ask questions regarding patient distress or instruct the patient to capture a cardiac rhythm strip on the wearable device.

In some embodiments, the protocol component comprises alarms to maximize the likelihood that the instructions will be followed and the system will be used optimally. For example, in some embodiments, the protocol component comprises system diagnostics and any identified anomaly is noted with an alarm. Alarms include audio, text, graphical or other desired warnings sent to the subject, the clinician device, and/or system manufacturer/distributor. In some embodiments, alarms are used to ensure proper compliance with instructions and/or protocols. For example, if the subject is not properly following the protocol, the display may indicate such and recommend the correct next step. Alternatively, if the subject fails to follow a protocol (e.g., fails to keep up with a colon cleansing protocol), a healthcare provider may be provided with an alarm so that the subject can be contacted (e.g., by phone, text, e-mail, etc.) to ensure that the subject stays compliant with the protocol.

In some embodiments, the protocol component comprises a means for which the subject can send questions separately or in conjunction with an image to a clinician via a clinician device which is in communication with the system.

The protocol component may be built on any desired hardware/software platform. In some embodiments, software components are provided via an application service provider (ASP) (e.g., are accessed by users within a web-based platform via a web browser across the internet; is bundled into a network-type appliance and run within an institution or an intranet: is provided as a software package and used as a stand-alone system; or is provided as downloadable software to an subject's device(s). The software components may be built on a system that comprises appropriate privacy and security features to comply with legal regulations regarding sharing and transferring of patient data and medical information.

e) Clinician Device

In some embodiments, a clinician device may be in contact and/or communication with the information processing device. The clinician device is in contact and/or communication with a clinician or other health care or health care-related worker (e.g., third party trained effluent evaluation experts may be employed). Collectively, any such party is referred to as a clinician herein. The clinician device allows the clinician to remain in contact with the subject during in-home prep, to monitor progress of the cleanse, or to answer any questions or address any concerns from the subject.

In some embodiments, the system generates an alert and/or report of a new bowel effluent image or inquiry generated by the subject to a clinician device. In some embodiments, the image of the bowel effluent, and optionally the background image, is sent to a clinician device in contact with a clinician using a communication device within or in electronic communication with the information processing component. In some embodiments, a clinician evaluates the image(s) included in the alert and/or report on the clinician device and makes a decision regarding the status of the colon cleanse. In some embodiments, the clinician communicates the decision on the status of the colon cleanse with the clinician device. In some embodiments, the clinician device relays messages in the form of instructions or status identifiers to the subject via the information processing component of the system. In some embodiments, the clinician device can alter or adjust the protocol component as a result of the decision. In some embodiments, the clinician communicates the answer to the inquiry to the subject with the clinician device. In some embodiments, the clinician communicates the answer to the inquiry to the subject via another means facilitated by the information processing component (e.g., phone, text, or email).

In some embodiments, the clinician device allows a clinician to know and/or determine the status of the colon cleanse prior to the subject appearing at the hospital or endoscopy clinic.

The clinician device may comprise any of a variety of computing devices configured to receive communications from the system, including but not limited to, a desktop computer, a mainframe computer, a laptop computer, a personal digital assistant (PDA), a portable computer (e.g., mobile devices such as telephones), a tablet computer (e.g., standard tablets, slates, mini tablets, phablets, customer handheld devices), and a wearable computer (e.g., eyeglass, wristwatch, clothing, helmet, etc.).

The clinician device may comprise a data collection, storage and analysis system. The data may be stored on the clinician device or on another device in communication with the clinician device, such as a remote secure server. This system may collect and analyze data on one patient over a single preparation to provide time course information of the preparation and allow retrospective review of the preparation by a clinician. This system may collect and analyze data on one patient over multiple preparations such that previous preparations can be used to design a starting protocol or analyzed to select protocols which resulted in the most effective previous preparation. This system may collect and analyze data for multiple patients such that protocols can be optimized for best practices and/or procedure outcomes. Data from multiple patients may be used to alter or design protocols for a patient's first colon preparation based on common medical history and/or risk factors for inadequate preps.

### 3. PREPARATION KITS

The present disclosure also provides colonoscopy preparation kits including one or more of: at least one purgative; a plurality of flushable marker; an anti-foaming agent; and computer readable medium, software, or an interface (e.g., an internet or computer application) for using the system described herein. Description provided elsewhere for purgatives, flushable markers, anti-foaming agents and the components of the system apply to the kit as well. In some embodiments, the kits further comprise containers to collect fecal samples or used toilet paper and/or wipes throughout the preparation.

### 4. EXAMPLES

### Example 1

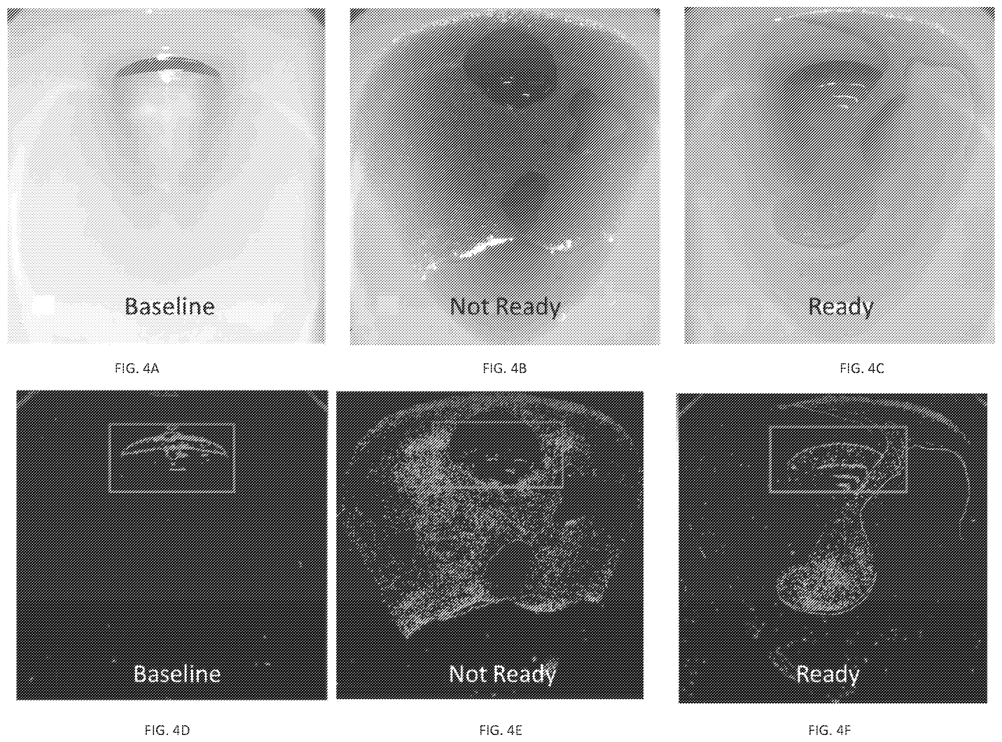

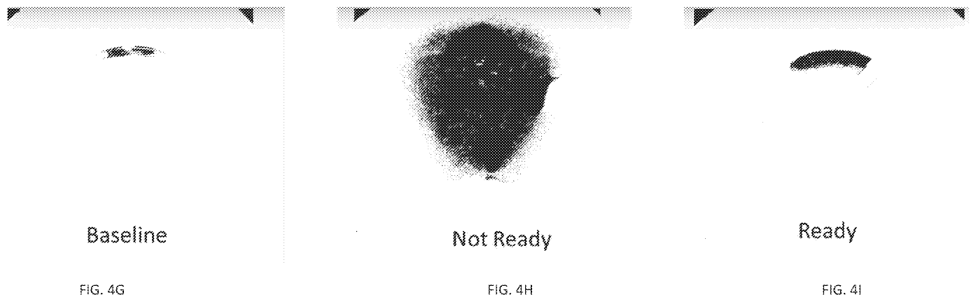

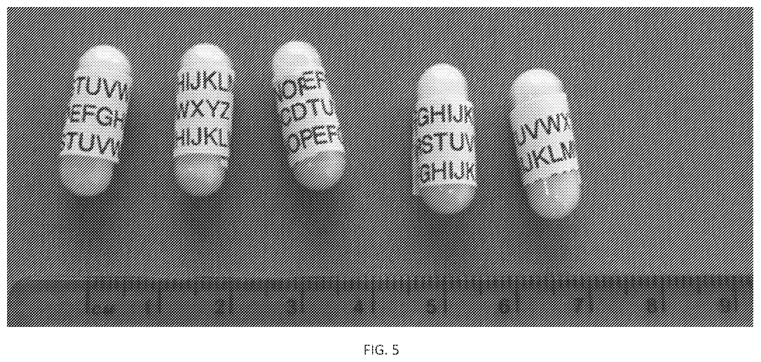

Colonoscopy preparation was performed under typical conditions in the home and images were captured with an Apple iPhone 10. FIG. 4A is a baseline image, as the preparation commenced. FIG. 4B is an exemplary final image where preparation was still deemed incomplete (Not Ready), whereas FIG. 4C was the earliest image where the preparation was sufficient (Ready), based on the judgements of a colonoscopy preparation expert. These images demonstrate the time point in the preparation where the preparation quality makes the transition from inadequate to adequate.

Image analysis using edge detection was applied to FIGS. 4A-4C, resulting in the images in FIGS. 4D-4F, respectively. The white edges in the box, capturing the bend in the bottom of the toilet, are apparent at Baseline (FIG. 4D). In the image of FIG. 4E, where the prep was NOT ready, these white edges cannot be seen. In the image of FIG. 4F, where the preparation was sufficient (Ready), the white edges in the box were seen once again.

Image analysis using red pixel intensity, where intensity values 125 to 255 are white, was applied to FIGS. 4A-4C, resulting in the images in FIG. 4G-4I, respectively. The water in the baseline image (FIG. 4G) had pixel values greater than 125, except where a shadow was cast on the bottom of the toilet. In the image of FIG. 4H, where the prep was NOT ready, much of the water present had a pixel value less than 125, shown in black. In the image of FIG. 4I, where the preparation was sufficient (Ready), the water had a red pixel intensity greater than 125, shown in white, easily distinguishable from the image of FIG. 4H.

### Example 2

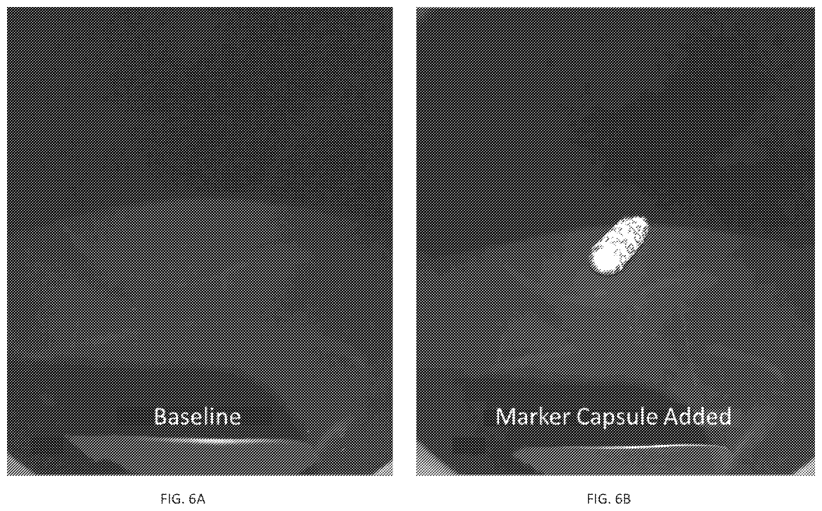

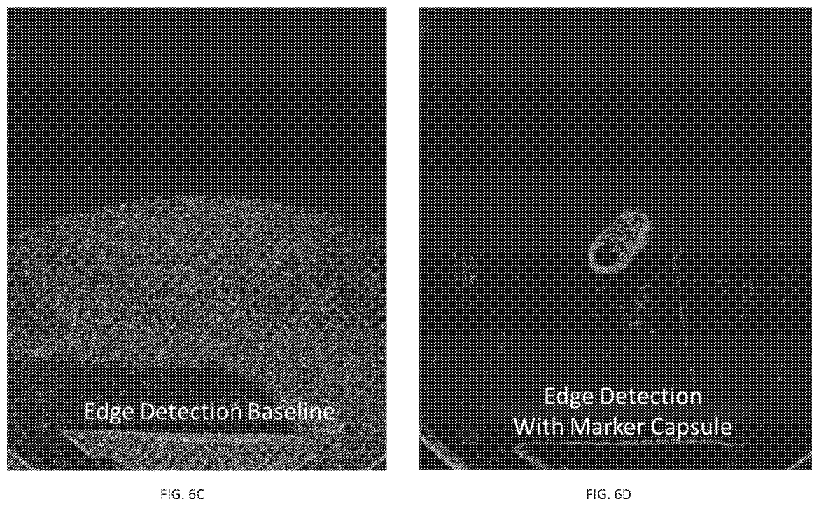

Several exemplary marker capsules are shown in FIG. 5. Other examples may differ in shape, color, and geometric design to optimize contrast in low-contrast settings, such as dark toilet bowls. The impact of a marker capsule on edge detection is shown in FIGS. 6A-6D. FIG. 6A shows an image of a dark toilet drain at baseline. Image analysis using edge detection was applied to FIG. 6A and no distinct edges were detected on the bottom of the drain (FIG. 6C). The addition of the marker capsule (FIG. 6B) provided substantial contrast at the bottom of the drain. Thus, when image analysis using edge detection was applied to FIG. 6B, multiple edges for image analysis were detected at the bottom of the drain (FIG. 6D).

It is understood that the foregoing detailed descriptions are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

1. A method of assessing status of a colon preparation in a subject comprising:
capturing an image of a bowel effluent in a toilet with an imaging device; and
assessing the image for: one or more aspects of the bowel effluent including color of effluent, clarity of effluent, presence of particulate matter or solid material, density of particulate matter or solid material, texture of the effluent, or combinations thereof; observable edges or contrasts in the toilet; or any combination thereof.

2. The method of claim 1, further comprising capturing a background image of the toilet.

3. The method of claim 1, further comprising loading the image, and optionally the background image, into an information processing component from the imaging device.

4. The method of claim 1, wherein the image of the bowel effluent, and optionally the background image, is sent to a clinician device in contact with a clinician using a communication device within or in electronic communication with the information processing component.

5. The method of claim 4, wherein the clinician sends messages via the clinician device informed by the image or images to the information processing component directly or indirectly via the communication device, and a protocol component of the information processing component is modified by the messages from the clinician device to display the status of the colon preparation to the subject.

6. The method of claim 1, wherein the information processing component assesses the image and modifies a protocol component to display a status of the colon preparation to the subject.

7. The method of claim 1, further comprising scoring the colon preparation with a pre-procedure preparation scale.

8. The method of claim 1, further comprising adding a flushable marker to the toilet.

9. The method of claim 8, wherein the observable edges or contrasts in the toilet comprise edges or contrasts along bottom of toilet bowl, edges or contrasts from the flushable marker, or a combination thereof.

10. (canceled)

11. The method of claim 1, wherein the method determines the status of a colon preparation in real time.

12. A system for assessing the status of a colon preparation in a subject, comprising:
an imaging device;
a communication component;
an information processing component configured to analyze data from two or more images of bowel effluent; and
a protocol component.

13-17. (canceled)

18. The system of claim 12, wherein the protocol component displays instructions for the colon preparation, provides the status of the colon preparation, or a combination thereof.

19. The system of claim 12, wherein the protocol component comprises an alarm protocol to alert the subject to an instruction or status of the colon preparation.

20. The system of claim 12, wherein the protocol component comprises a query function to question the subject regarding symptoms or side effects or instruct the subject to capture additional physiological data.

21. The system of claim 20, wherein the information processing component or the protocol component alters protocols based on responses to the query function.

22. The system of claim 12, wherein the information processing component is in communication with a clinician device in contact with a clinician.

23. (canceled)

24. The system of claim 12, wherein the information processing component comprises image analysis software to analyze images received from the imaging device.

25. The system of claim 24, wherein the information processing component alters protocols based on the image analysis.

26. The system of claim 24, wherein the information processing component assigns a score to the colon preparation based on a pre-procedure preparation scale.

27-29. (canceled)

30. A colonoscopy preparation kits comprising one or more of:
at least one purgative;
a plurality of flushable markers;
an anti-foaming agent; and
computer readable medium, software, or interface instructions for using the system of claim 12.
